SEQUENCE LISTING (2 sequences)

SEQ ID NO: 1 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 2 - protein, 5 aa, Artificial Sequence
  Description of Artificial Sequence Synthetic peptide
FLFLF

(19) United States
(12) Patent Application Publication
(10) Pub. No.: US 2006/0008891 A1
(43) Pub. Date: Jan. 12, 2006

(54) Cathepsin G-related peptides as modulators of formylpeptide receptors (FPR)

(75) Inventors: Ji Ming Wang, Frederick, MD (US); Ronghua Sun, Frederick, MD (US); Joost J. Oppenheim, Frederick, MD (US); Thomas Rogers, Philadelphia, PA (US)

Correspondence Address: NATIONAL INSTITUTES OF HEALTH;C/O VENABLE LLP, P. O. BOX 34385, WASHINGTON, DC, 20043-9998, US

(73) Assignee: The Government of USA as repersented by the Secretary, Department of Health and Human Services, Rockville, MD (US)

(21) Appl. No.: 11/154,744

(22) Filed: Jun. 17, 2005

Related U.S. Application Data
(60) Provisional application No. 60/615,607, filed on Oct. 5, 2004.
(60) Provisional application No. 60/581,765, filed on Jun. 23, 2004.

(51) Int. Cl. C12N 9/64

(52) U.S. Cl. 435/226; 530/350

(57) ABSTRACT

The present invention relates, e.g., to a method for identifying a peptide of CaG, or an active variant thereof, that modulates a CaG-FPR response (e.g., chemotaxis), comprising screening a candidate CaG peptide or active variant thereof for its ability to modulate a CaG-FPR induced response. Methods for identifying peptide mimetics of CaG, peptides that are more potent agonists of FPR than is CaG, and antagonists of FPR are described.

This application claims the benefit of the filing dates of provisional application 60/581,765, filed Jun. 23, 2004, and U.S. provisional application 60/615,607, filed Oct. 5, 2004, both of which are hereby incorporated by reference in their entirety.

### FIELD OF THE INVENTION

The present invention relates, e.g., to methods for identifying peptides of cathepsin G (CaG), or active variants thereof, that modulate activities of the receptor, FPR, including chemotactic behavior.

### BACKGROUND INFORMATION

Neutrophils rapidly accumulate at sites of inflammation and infection, and these cells are activated by bacterial and host-derived factors. Following phagocytosis of microorganisms or other particulate substances, neutrophils secrete a variety of mediators that possess potent proinflammatory and antimicrobial activities. These mediators include a group of antibiotic peptides and proteases that are stored in neutrophil granules and released during the process of degranulation.

One such mediator is cathepsin G (CaG), which is a member of the serine protease family, is largely present in azurophilic granules of neutrophils, and accounts for up to 18% of the azurophil granule proteins. It is referred to as a chymotrypsin-like enzyme because it hydrolyzes peptide bonds after leucine, methionine and phenylalanine residues. In addition to its capacity to proteolytically degrade engulfed cell debris and its microbicidal activity, CaG displays a variety of pathophysiological effects, such as degradation of extracellular matrix, regulating bioactivity of cytokines and cytokine receptors, induction of proinflammatory cytokines in macrophages, enhancing hematopoietic progenitor cell mobilization by cleaving CD106, disrupting interaction between CXCR4/SDF-1α, and regulation of neutrophil migration by modifying the P-selectin receptor PSGL-1. Human CaG has been shown to promote specific antibody responses when injected in mice. In addition, CaG has been implicated in supporting wound healing as shown by decreased wound breaking strength in mice depleted of CaG gene. CaG purified from human neutrophil granules was reported to be a potent chemoattractant for human phagocytic leukocytes and to increase the random mobility of T lymphocytes in vitro. The chemotactic activity of CaG for phagocytic leukocytes was reported to be sensitive to pertussis toxin, implying the involvement of a G protein coupled seven-transmembrane (STM) receptor.

The inventors show herein that the receptor used by the ligand CaG in at least some of its physiological manifestations is the Gi protein coupled human STM (seven transmembrane) receptor, formylpeptide receptor (FPR); and they identify unexpected properties of this receptor/ligand interaction.

### DESCRIPTION OF THE DRAWINGS

FIG. 1 shows that CaG induces human monocyte migration by interacting with a formyl peptide receptor FPR. FIG. 1A shows a comparison of migration of monocytes in response to CaG, the bacterial peptide fMLF and MMK-1, a synthetic peptide specific for FPRL1. The results are expressed as the mean number of migrating cells (±SD) in 3 high power fields (HPFs). * Denotes statistically significant increase in cell migration induced by stimulants versus medium control (P<0.01). FIG. 1B shows the attenuation of monocyte chemotaxis. Monocytes were pre-incubated with medium alone, fMLF (100 nM), medium containing 0.05% DMSO (as a solvent for MMK-1), or MMK-1 (5 μM) at 37° C. for 30 min. After extensive washing, the chemotactic responses of the cells to fMLF (10 nM), MMK-1 (100 nM) or CaG (10 μg/ml, 400 nM) were measured. * Indicates significantly reduced cell migration in response to stimulants as compared to cells treated with medium alone or with medium containing 0.05% DMSO(P<0.001). FIG. 1C shows the attenuation of monocyte chemotaxis to fMLF by CaG. Monocytes preincubated with 10 μg/ml for 30 min at 37° C. were tested for chemotactic response to different concentrations (Log molar) of fMLF. Significantly reduced migration as compared with cells treated with medium alone was indicated by *. FIG. 1D shows the blockade of CaG-induced monocyte chemotaxis by anti-FPR antibody. Monocytes were pre-incubated with different concentrations of anti-FPR at room temperature for 15 min and then were evaluated for chemotactic response to CaG (10 μg/ml), fMLF (10 nM), or MMK-1 (100 nM). * Indicates significantly reduced chemotactic response as compared with cells treated with normal mouse IgG. FIG. 1E shows the inhibition of CaG-induced monocyte migration by CsH. Monocytes mixed with different concentrations of CsH were assessed for their chemotactic response to CaG (10 μg/ml), fMLF (10 nM), or MMK-1 (100 nM). * Indicates significantly decreased chemotactic response as compared to cells treated with medium alone (P<0.01).

FIG. 2 shows that CaG is chemotactic for RBL cells transfected with FPR (ETFR cells). FIG. 2A shows the migration of ETFR cells in response to different concentrations of CaG, fMLF, or MMK-1. * Indicates significantly increased chemotactic response as compared to response to medium alone (P<0.01). FIG. 2B shows the inhibition of CaG-induced ETFR cell migration by anti-FPR antibody. ETFR cells were pre-incubated with different concentrations of anti-FPR antibody or control IgG at room temperature for 15 min and then were examined for their chemotactic response to CaG (10 μg/ml). Significantly reduced migration of anti-FPR antibody treated cells in comparison with IgG-treated cells is indicated by * (P<0.01). FIG. 2C shows the inhibition of ETFR cell chemotaxis by CsH. ETFR cells were mixed with designated concentrations of CsH and their chemotactic response to CaG (10 μg/ml) or fMLF (100 nM) was assessed. * indicates significantly reduced migration of CsH-treated cells compared to cells treated with medium alone (P<0.01). HPF, High power field.

FIG. 3 shows the effect of CaG on Ca²⁺ mobilization in phagocytic leukocytes and ETFR cells in response to fMLF. Cells loaded with Fura-2 were examined for Ca²⁺ mobilization in response to CaG (10 μg/ml) and fMLF. CaG failed to induce significant Ca²⁺ flux in monocytes (FIG. 3A), or neutrophils (FIG. 3B), but induced a measurable level of Ca flux in ETFR cells (FIG. 3C). CaG (10 μg/ml) significantly attenuated cell response to 0.1 nM fMLF (FIGS. 3A, 3B, 3C) and in ETFR cells the cell response to CaG (10 μg/ml) was attenuated by 0.1 nM fMLF (FIG. 3C). RFU: relative fluorescence units.

FIG. 4 shows the effect of CaG on the cell surface expression of FPR and on binding of ³[H]-fMLF. ETFR cells (FIG. 4A(1)) or monocytes (FIG. 4A(2)) (1×10⁶) were pretreated with medium alone, 5 μg/ml CaG, or 1 μM fMLF at 37° C. for 30 min. The cells were washed and stained with a FITC conjugated mouse anti-human FPR antibody (α-FPR) or with an isotype matched control IgG (IgG). Cell surface expressed FPR was then detected by FACS analysis. The internalization of FPR induced by fMLF (1 μM) or different concentrations of CaG was assessed by incubating ETFR cells on chamber slides with the ligands for 30 min at 37° C. The cells were then washed, stained with anti-FPR antibody followed by a FITC conjugated secondary antibody, and examined for fluorescence distribution with confocal microscopy (FIG. 4B). FIG. 4B(1) shows medium; FIG. 4B(2) shows fMLF at 1 μM; FIGS. 4A(3) through 4A(6) show CaG at 1 μg/ml, 21 μg/ml, 5 μg/ml, and 10 μg/ml, respectively. ETFR cells and monocytes (FIG. 4C(1) and 4C(2), respectively) were also measured for binding of ³[H]-fMLF and competition by unlabeled fMLF or CaG. The total binding was determined by using 3 nM ³[H]-fMLF on 2×10⁶ cells. The nonspecific binding was assessed by residual radioactivity (cpm) on cells in the presence of 10 μM unlabeled fMLF. The results are expressed as the percentage inhibition of specific binding by different concentrations of unlabeled fMLF or CaG. The IC50s for fMLF and CaG were estimated at 3 nM and 100 nM, respectively. ETFR cells (FIG. 4D(1)) or the parental RBL cells (FIG. 4D(2)) were measured for binding of ¹²⁵I-CaG (25 nM) in the presence or absence of unlabeled CaG or fMLF. The number of binding sites per cell and Kd were estimated with a LIGAND computer program.

FIG. 5 shows the activation of MAPKs and Akt by CaG. ETFR cells (FIGS. 5A, 5C, 5D) or parental RBL cells (FIG. 5B) were plated in Petri dishes and grown for 48 h. After culture in serum-free medium overnight, the cells were stimulated with 10 μg/ml CaG for different time periods, or with different concentrations of CaG for 10 min at 37° C. Cell lysates were subject to SDS-PAGE and Western blotting with specific anti-p-ERK1/2 (FIGS. 5A, 5B), p-p38 MAPK (FIG. 5C), or p-Akt (FIG. 5D) antibodies. The stripped membranes were subsequently analyzed to determine total ERK1/2, p38 MAPK or Akt protein levels. fMLF at 100 nM was used in control experiments.

FIG. 6 shows the translocation of PKCζ induced by CaG in ETFR cells. ETFR cells (FIG. 6A) or parental RBL cells (FIG. 6B) were treated with medium, 100 nM fMLF, or 10 μg/ml CaG at 37° C. for 10 min. The cells were fixed with 4% PFA and were separately stained with anti-PKCα/β, δ, or ζ antibody. PMA (500 nM), an activator of DAG-calcium dependent PKC isozymes, was used as a positive control, which induced translocation of PKCζ, PKCα/β, and PKCδ in ETFR and RBL cells.

FIG. 7 shows the effect of protein kinase inhibitors on CaG-induced cell migration. ETFR cells (FIGS. 7A, 7B, 7C) or neutrophils (FIGS. 7D, 7E) were incubated with different concentrations of chelerythrine (FIGS. 7C, 7D), Gö6850 (FIG. 7E), Staurosporine (FIG. 7A), or Wortmannin (FIG. 7B) at 37° C. for 1 h. The cells were then examined for chemotaxis in response to CaG (10 μg/ml) or fMLF (100 nM). * Indicates significantly reduced cell migration compared to cells treated with medium alone (P<0.01). None of the inhibitors at concentrations used in the studies affected spontaneous cell migration.

FIG. 8 shows that CaG treatment of ETFR cells did not generate soluble chemotactic activity. ETFR and RBL cells (10×10⁶/ml) were incubated with medium or 10 μg/ml CaG at 37° C. for 1 h. Supernatants were collected and measured for chemotactic activity for un-treated ETFR cells. The supernatants were also treated for 15 min at room temperature with α1-ACT (0.75 μM) before being tested for chemotactic activity for ETFR cells. α1-ACT treated fMLF (100 in M) was tested in parallel. ETFR+med: supernatant from ETFR cells treated with medium; ETFR+CaG: supernatant from ETFR cells treated with CaG; RBL+med and RBL+CaG: supernatants from RBL cells treated with medium or CaG. fMLF (100 nM) and CaG (10 μg/ml) cultured in the absence (ACT −) or presence (ACT +) of α1-ACT were used as positive controls. * Indicates significantly reduced chemotactic activity shown by α1-ACT-treated CaG and supernatants compared to medium treatment.

FIG. 9 shows that the N-terminal sequences of FPR are not required for the chemotactic activity of CaG. FIG. 9A is a cartoon showing the chimeric receptor CHI-39, in which the N-terminal sequences from FPR are substituted with comparable sequences from the N-terminus of human FPRL1, a receptor not activated by CaG. FIG. 9B shows chemotactic activity in response to CaG of HEK293 cells transfected with FPR (left side of figure) or transfected with CHI-39 (right side of figure).

### DESCRIPTION OF THE INVENTION

The present inventors identify formylpeptide receptor (FPR) as the receptor activated by the ligand, cathepsin G (CaG), and identify some unexpected properties of this receptor/ligand interaction. For example, although CaG induces chemotaxis of phagocytic cells expressing FPR, in other respects it acts only as a partial agonist for FPR. More particularly, CaG selectively activates PKC ζ, but not PKC α or PKC β, in the cell; and it does not elicit other activities which are exhibited by full FPR agonists, including a substantial induction of calcium flux.

The effects of CaG on FPR-expressing cells differ from the effects induced by the full FPR agonist, fMLF (a bacterial chemotactic peptide shown previously to bind to FPR at high affinity and to induce G-protein coupled signal transduction). Compared to fMLF, CaG appears to possess lower affinity for FPR, as evidenced by the requirement of 5-10 μg/ml (200-400 nM) of the protein to elicit optimal cell migration. Also, unlike fMLF, CaG does not trigger potent Ca²⁺ transients in phagocytic leukocytes or FPR transfected RBL cells and, despite its rapid effect on MAPK phosphorylation, there is a certain degree of delay (10-15 min) for CaG to reach its maximal activity as compared with fMLF. Furthermore, fMLF more potently desensitizes CaG-induced cell chemotaxis as compared with the capacity of CaG to attenuate cell response to fMLF. A lower capacity of CaG to activate FPR as compared to fMLF is also reflected by the finding that fMLF induces translocation of PKCα/β and ξ, members of the classical and atypical PKC subfamilies, respectively, whereas CaG only induces the translocation of PKCξ, an atypical PKC crucial for neutrophil chemotaxis.

These observations provide a basis for, e.g., the development of screening methods for agents, such as peptides of CaG, or active variants thereof, that mimic CaG (e.g., mimic the ability of CaG to act as a partial agonist for FPR, including the ability to induce chemotaxis of cells, such as phagocytes, expressing FPR); that act as more effective FPR agonists than CaG; or that act as antagonists of FPR. The invention can also be applied to experimental studies, e.g. studies designed to better understand the biological role of this neutrophil granule protein in host response in inflammation and microbial infection.

This invention relates, e.g., to a method for identifying a peptide of cathepsin G (CaG), or an active variant thereof, that modulates a CaG-FPR induced activity (response), comprising contacting a candidate peptide or active variant thereof with a cell comprising an FPR receptor, and determining the effect of the peptide on the receptor, e.g. determining if the peptide modulates one or more CaG-FPR induced activities (responses) in the cell. As used herein, the term a “CaG-FPR induced activity (response)” refers to one or more of the FPR-mediated activities (responses) that can be induced by CaG. As discussed in more detail elsewhere herein, among the FPR-mediated activities induced by CaG are the selective activation of PKCζ, but not PKCα or PKCβ; the induction of little or no calcium flux; the induction of chemotaxis of the cell; and the activation of MAP kinases. These various CaG-FPR induced activities may be induced by different domains of the CaG polypeptide; therefore, CaG peptides from different portions of the CaG polypeptide might be expected to induce different subsets of the activities. A peptide that modulates a CaG-FPR induced response can, for example, induce an FPR-mediated response to a greater degree than does CaG. That is, the peptide can act as a more potent agonist of FPR than does FPR. Alternatively, the peptide may modulate a CaG-FPR induced response by acting as an antagonist of CaG. The ability of a peptide to “modulate” a response, as used herein, includes to increase or to decrease the level of the response compared to the response elicited by CaG.

One aspect of the invention is a method for identifying a peptide of CaG, or an active variant thereof, that mimics CaG, comprising screening a candidate peptide for its ability, when contacted with an FPR-expressing cell, to selectively activate PKC ζ, but not PKC α and/or PKC β, in the cell; and/or screening the candidate for its lack of ability to induce substantial amounts of calcium flux. Alternatively, or in addition, the candidate may be screened for its ability to induce chemotaxis of the cell, and/or to activate a MAP kinase.

Another aspect of the invention is a method for identifying a peptide of CaG, or an active variant thereof, that is a more potent agonist of FPR than is CaG, comprising screening the candidate peptide for its ability, when contacted with an FPR-expressing cell, to
a) activate PKC ζ, PKC α and/or PKC β to a greater degree than does CaG; and/or
b) induce a greater amount of calcium flux of the cell than does CaG; and/or
c) induce a greater amount of chemotaxis than does CaG; and/or
d) activate a MAP kinase to a greater degree than does CaG.

Another aspect of the invention is a method for identifying a peptide of cathepsin G, or an active variant thereof, that is an antagonist of FPR, comprising screening the candidate peptide for its ability, when contacted with an FPR-expressing cell, to antagonize an agonist of FPR (such as CaG or fMLF). For example, the candidate can be tested for its ability to antagonize the ability of the agonist to:
a) activate PKC ζ, PKC α and/or PKC β in the cell; and/or
b) induce chemotaxis; and/or
c) activate a MAP kinase.
   In a preferred embodiment, the candidate antagonist is screened for its ability inhibit substantially all activation of PKC ζ, PKC α and PKC β, chemotaxis and/or activation of a MAP kinase by the agonist.

Another aspect of the invention is a method for identifying an agent (e.g., a small molecule) which enhances the agonist ability of CaG on FPR, comprising screening a candidate agent for its ability, when contacted with an FPR-expressing cell, in the presence of CaG, to
a) activate PKC ζ, PKC α and/or PKC β to a greater degree than does CaG; and/or
b) induce a greater amount of calcium flux of the cell than does CaG; and/or
c) induce a greater amount of chemotaxis than does CaG; and/or
d) activate a MAP kinase to a greater degree than does CaG.

Another aspect of the invention is a method for identifying an agent (e.g., a small molecule) which inhibits the agonist ability of CaG on FPR, comprising screening a candidate agent for its ability, when contacted with an FPR-expressing cell, in the presence of CaG, to
a) activate PKC ζ, PKC α and/or PKC β to a lesser degree than does CaG; and/or
b) induce a lower amount of chemotaxis than does CaG; and/or
c) activate a MAP kinase to a lesser degree than does CaG.

In embodiments of the methods of the invention, the CaG-FPR induced response comprises the induction (or inhibition) of chemotactic activity of a human phagocyte. Any method of the invention may be a high throughput method.

Peptides of the invention that act as CaG mimetics or as agonists that exhibit stronger FPR agonist activity than CaG, may stimulate any FPR-mediated response that is mediated by CaG. Among such responses are, e.g., an proinflammatory response, an antimicrobial response, an antibody response, wound healing, increased phagocytosis, or production of reactive oxygen intermediates.

Peptides of the invention that act as FPR antagonists may inhibit any undesirable FPR-mediated response that is mediated by CaG. For example, the peptide may inhibit an undesirable inflammatory response.

In one embodiment of the invention, the method comprises
contacting a cell that expresses FPR or an active fragment, variant, mimetic or analog of FPR (e.g., a eukaryotic cell that has been transfected with a construct such that the cell over-expresses FPR) with a candidate peptide, under conditions effective for the candidate peptide to exert an effect on a CaG/FPR-mediated activity; and
determining the level of that activity in the contacted cell compared to a baseline value. The term “a fragment, variant, mimetic or analog” of FPR includes any of a variety of modified FPR molecules, provided that the modified protein retains at least one of the activities (e.g., the ability to be activated by CaG) of the wild type protein. Modified proteins can take the form of, e.g., conservative amino acid substitutions, deletions, additions, etc, and include naturally occurring allelic variants. Suitable types of modified proteins will be evident to the skilled worker.

Another aspect of the invention is a kit for carrying out any of the methods of the invention, comprising an FPR-expressing cell and means for detecting one or more elements of a CaG-FPR induced response (e.g., partial agonist response). More particularly, the kit comprises means for measuring activation of PKC ζ, PKC α and/or PKC β and/or for measuring calcium flux; and/or for measuring chemotaxis and/or activation of a MAP kinase.

A “peptide of CaG,” as used herein, refers to a peptide containing a contiguous amino acid sequence from a full-length CaG polypeptide. Preferably, the CaG is human. However, under some conditions, peptides of CaG from other sources, such as mouse, may also be used. A peptide of CaG (sometimes referred to as a “CaG peptide”) identified by a method of the invention can be from any portion of CaG, and can be of any length, provided that the peptide is effective to mimic or modulate (e.g., induce or inhibit) one or more CaG/FPR responses (e.g., the peptide can mimic a CaG-mediated response, or is a better FPR agonist than CaG, or is an antagonist of FPR). In some embodiments of the invention, the peptide can modulate only one FPR-mediated activity; in other embodiments, the peptide can modulate as many as all of the FPR-mediated activities induced by CaG. In a preferred embodiment, the peptide modulates the chemotactic behavior of CaG toward FPR-expressing cells.

Preferably, a peptide of the invention is from about 15 to about 25 amino acids in length (e.g. about 20 amino acids), although larger and smaller peptides are acceptable, provided they exhibit the desired properties.

As used herein, an “isolated” peptide is one that is in a form not found in its original environment or in nature, e.g., more concentrated, more purified, separated from at least one other component with which it is naturally associated, in a buffer, etc.

Methods of producing peptides are conventional in the art. For example, a CaG peptide can be chemically synthesized, or it can be cleaved from larger polypeptide, such as full-length CaG, using enzymatic or chemical digestion procedures. Alternatively, a nucleic acid encoding a peptide can be cloned and the peptide expressed recombinantly. Combinations of these methods can also be used.

Methods of making recombinant constructs, in which a sequence encoding a peptide of interest is operatively linked to an expression control sequence, are conventional. Methods of making recombinant constructs, as well as many of the other molecular biological methods used in conjunction with the present invention, are discussed, e.g., in Sambrook, et al. (1989), Molecular Cloning, a Laboratory Manual, Cold Harbor Laboratory Press, Cold Spring Harbor, N.Y.; Ausubel et al. (1995). Current Protocols in Molecular Biology, N.Y., John Wiley & Sons; Davis et al. (1986), Basic Methods in Molecular Biology, Elseveir Sciences Publishing, Inc., New York; Hames et al. (1985), Nucleic Acid Hybridization, IL Press; Dracopoli et al. Current Protocols in Human Genetics, John Wiley & Sons, Inc.; and Coligan et al. Current Protocols in Protein Science, John Wiley & Sons, Inc.

The identification of a peptide that elicits a CaG/FPR induced activity can allow for the identification of a domain of CaG that is involved in one or more of the diverse activities of the protein. Once identified, a peptide corresponding to a domain of interest can be further modified, for example to enhance its therapeutic effect and/or to reduce its toxic properties.

If a CaG peptide is demonstrated to exhibit a promising activity, it can be further modified, e.g. by adding one or more amino acids to one or both ends of the peptide, according to the sequence found in full-length CaG, and then tested for improved activity. Alternatively, one or more of the amino acid residues of the peptide can be varied (e.g., substituted or deleted) and one or more amino acid residues can be added at any position in the peptide. The sequence of human CaG, which can be used to aid in designing modified peptides, is: MQPLLLLLAFLLPTGAEAGEIIGGRESRPHSRPYMAYLQIQSPAGQSRCGGFLVREDFVLTA AHCWGSNINVTLGAHNIQRRENTQQHITARRAIRHPQYNQRTIQNDIMLLQLSRRVRRNRN VNPVALPRAQEGLRPGTLCTVAGWGRVSMRRGTDTLREVQLRVQRDRQCLRIFGSYDPRR QICVGDRRERKAAFKGDSGGPLLCNNVAHGIVSYGKSSGVPPEVFTRVSSFLPWIRTTMRSF KLLDQMETPL (SEQ ID NO: 1). Typical active variants of CaG peptides are discussed below.

Methods of isolating and purifying peptides are conventional in the art. For example, methods of harvesting and isolating (e.g., purifying) a peptide, e.g. from a cell expressing a suitable recombinant construct, include protein chromatographic methods, including ion exchange, gel filtration, HPLC, and immunoaffinity chromatography. Other methods will be evident to the skilled worker.

An “active variant” of a CaG peptide, as used herein, includes a peptide that is substantially identical to a CaG peptide and that retains an activity (such as the ability to modulate a CaG-FPR response, e.g. chemotaxis) of the CaG peptide from which it is derived. As used herein, the singular forms “a,” “an,” and “the” include plural referents unless the context clearly dictates otherwise. For example, “an” activity, as used above, means one or more activities. A skilled worker can readily test a variant to determine if it is active, using conventional, routine procedures.

In one embodiment, an active variant of a CaG peptide is substantially identical to the comparable peptide from its wild type counterpart. By “substantially identical” is meant herein that the amino acid sequences are at least about 75% identical (e.g., at least about 80%, 85%, 90, 95%, 97% or 99% identical.). The degree of sequence identity may be optimized by sequence comparison and alignment algorithms known in the art (see Gribskov and Devereux, Sequence Analysis Primer, Stockton Press, 1991, and references cited therein) and calculating the percent difference between the amino acid sequences by, for example, the Smith-Waterman algorithm as implemented in the BESTFIT software program using default parameters (e.g., University of Wisconsin Genetic Computing Group).

An active variant peptide can comprise, e.g., one or more conservative or non-conservative amino acid substitutions, deletions, additions, insertions, inversions, fusions or truncations, or a combination of any of these. The variations may be introduced into a peptide artificially. Alternatively, peptides derived from naturally occurring sequence variations (e.g., allelic variants) of CaG, due to genetic mutation, polymorphisms, evolutional divergence, etc., are also included. Active variants included homologs, muteins, analogs and derivatives. Suitable types of variants will be evident to the skilled worker.

A preferred group of active variants of CaG are those in which at least one amino acid residue and preferably, only one, has been substituted by a different residue. The types of substitutions that may be made in the protein molecule include conservative substitutions, which may include exchanges within one of the following five groups:

| | | |
|---|---|---|
| 1 | Small aliphatic, nonpolar or slightly | Ala, Ser, Thr (Pro, Gly); |
| | polar residues | |
| 2 | Polar, negatively charged residues | Asp, Asn, Glu, Gln; |
| | and their amides | |
| 3 | Polar, positively charged residues | His, Arg, Lys; |
| 4 | Large aliphatic, nonpolar residues | Met, Leu, Ile, Val (Cys) |
| 5 | Large aromatic residues | Phe, Tyr, Trp. |

The three amino acid residues in parentheses above have special roles in protein architecture. Gly, the only residue lacking a side chain, imparts flexibility to the chain. Pro, because of its unusual geometry, tightly constrains the chain. Cys can participate in disulfide bond formation which is important in protein folding.

More substantial changes in biochemical, functional properties are made by selecting substitutions that are less conservative, such as between, rather than within, the above five groups. Such changes may differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of a peptide or protein, e.g. in terms of its proteolytic activity. However, when it is difficult to predict the exact effect of the substitution, deletion or insertion in advance of doing so, one skilled in the art will appreciate that the effect can be evaluated by routine screening assays such as those described here, without requiring undue experimentation.

An active variant of the invention must possess a CaG/FPR activity. In view of this functional requirement, use of proteins homologous to CaG from other host derived species (e.g., mouse) and genera, as well as from plants, bacteria or animal sources, including proteins not yet discovered, fall within the scope of the invention if these proteins have sequence homology and the recited biochemical and biological activity.

CaG peptides and active variants thereof are sometimes referred to herein as “CaG-related peptides.”

Other active variants of the invention include “chemical derivatives” of CaG-related peptides. Such chemical variants contain additional chemical moieties not normally a part of the peptide. Covalent modifications of the peptide are included within the scope of this invention. Such modifications may be introduced into the molecule by reacting targeted amino acid residues with an organic derivatizing agent that is capable of reacting with selected side chains or terminal residues. Such chemically modified and derivatized moieties may improve the peptide's solubility, absorption, biological half life, and the like. These changes may eliminate or attenuate undesirable side effects of the peptide in vivo. Modifications or variations can occur anywhere in a polypeptide, including the peptide backbone, the amino acid side-chains and the amino or carboxyl termini.

Moieties capable of mediating such effects are disclosed, for example, in Remington's Pharmaceutical Sciences, Mack Publishing Company, Easton Pa. (Gennaro 18th ed. 1990). Known peptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, PEGylation, proteolytic processing, phosphorylation, prenylation, racemization, selenoylation, sulfation, transfer-RNA mediated addition of amino acids to proteins such as arginylation, and ubiquitination.

Other active variants of the invention include fusion peptides, in which the peptide is fused with another compound, such as a compound to increase the half-life of the peptide (for example, polyethylene glycol), or in which additional amino acids are fused to the peptide, such as a leader or secretory sequence or a sequence which is employed for purification of the peptide, commonly for the purpose of creating a genetically engineered form of the peptide that is susceptible to secretion from a cell, such as a transformed cell. Other active variants include peptides in which one or more of the amino acid residues includes a substituent group.

Active variants of CaG peptides can be generated by methods similar to those used to produce CaG peptides. For example, the variants can be synthesized chemically, to generate a particular variant of interest, to contain random or defined amino acid substitutions at a designated position in the peptide, etc. Alternatively, nucleic acids encoding CaG variants can be cloned and expressed. In one embodiment, nucleic acids comprising random or defined variations at a designated position in a CaG peptide-encoding sequence are generated by chemical methods and are then cloned, expressed and tested with methods of the invention. In another embodiment, nucleic acids encoding CaG are modified by art-recognized site-specific mutagenesis methods, either before or after cloning and expressing the encoded peptides.

The term, a “CaG/FPR induced response,” as used herein, refers to one or more of the responses induced by contacting an FPR-expressing cell with CaG. Among the wide variety of such responses are: protease activity (e.g., the capacity to proteolytically degrade engulfed cell debris and to degrade extracellular matrix); antimicrobicidal activity; regulation of bioactivity of cytokines and cytokine receptors (e.g., increase IL-8 production by epithelial cells, and induction of proinflammatory cytokines in macrophages); enhancing hematopoietic progenitor cell mobilization by cleaving CD106; disrupting interaction between CXCR4/SDF-1a; regulation of neutrophil migration by modifying the P-selectin receptor PSGL-1; promotion of antigen-specific antibody responses (when injected in mice); supporting wound healing; potent chemoattractant for human phagocytic leukocytes and to increase the random mobility of T lymphocytes in vitro; mast cell degradation; decrease in glucocortocoid production; regulation of complement activation; mediating chemotaxis; release of antibacterial mediators such as reactive oxygen and nitrogen intermediates; activation (e.g., translocation) of PKCs; activation of nuclear factor (NF)kB; enhanced nuclear translocation of hypoxia-inducible factor-1a (HIF-1α); the production of biologically active vascular endothelial growth factor (VEGF); promotion of wound healing, and stimulation of glioma tumor growth and invasion.

Preferably, the CaG/FPR induced responses that are screened for in methods of the invention include one or more of: induction of chemotaxis of the FPR-expressing cell; selective activation of PKC ζ, but not substantially of PKC α or PKC β, in the cell; lack of induction of substantial amounts of calcium flux; and activation of a MAP kinase (one or more MAP kinases). This aggregate of responses represents a partial agonist response. In one embodiment of the invention, selective activation of PKC ζ, but not substantially of PKC α and/or PKC β is screened for. Preferably, a peptide is screened for its ability to induce chemotaxis.

The ability of a peptide candidate to induce an FPR-mediated activity can be tested (screened) in any FPR-expressing cell, including naturally occurring cells that express FPR, such as, e.g., leukocytes (e.g., mononuclear leukocytes, phagocytic leukocytes), human myeloid cells and human glioma cells; and cells from established cell lines that have been transfected to express FPR, such as, e.g., the rat basophilic leukemia cells (RBL) cells which have been transfected with FPR cDNA, called ETFR cells, or HEK293 cells transfected with FPR cDNA, such as the cells which are described in the Example VIII. Cells that have been transfected with active portions of FPR can also be used in assays of the invention. For example, cells transfected with FPR lacking N-terminal sequences, as described in Example VIII, can be used to assay for chemotactic activity.

According to the present invention, a CaG-related peptide exerts its action by recognizing a specific amino acid sequence present in FPR. Thus, methods described herein as targeting FPR can be carried out similarly without undue experimentation and with the same expected effect using a CaG-related peptide active on any other FPR family member. Suitable FPR family members include FPR from, for example, P. troglodytes, G. gorilla, P. Pygmaeus, M. mulatto, or S. Oedipus. Among the suitable FPR family members are human FPRL1 and FPRL2, and mouse FPR1 and FPR2.

With regard to the induction of chemotaxis: The ability of a candidate peptide to modulate FPR-mediated chemotaxis can be tested with conventional procedures, such as the methods described in the Examples herein. For example, low amounts of calcium (e.g., in the 4-400 nM range) are expected to induce chemotaxis in monocytes, neutrophils, ETFR cells, or HEK/FPR cells which are contacted with a CaG peptide of the invention. As shown in Example VIII, the N-terminal sequences of FPR are not required for the chemotactic activity of CaG. Therefore, a cell comprising an FPR receptor lacking these N-terminal sequences, or comprising a chimeric receptor such as CHI-39 described in the Example, can also be used in an assay for peptides that modulate FPR-mediated chemotaxis.

With regard to the selective activation of certain PKC isozymes: The Examples herein show that CaG substantially activates the atypical PKC isozyme, PKC ζ. “Substantial” activation, as used herein, refers to activation in the nanomolar to low micromolar range of a ligand. “Activation” of a PKC, as used herein, includes, e.g., the translocation of the PKC from the cytoplasm to the cell membrane, as is demonstrated for PKC ζ in the Examples. In some embodiments, the activation also comprises phosphorylation of the protein. In contrast to its effects on PKC ζ, the Examples show that CaG does not induce detectable amounts of PKC α or PKC β activation (by the sensitivity of the standard assay methods employed in the Examples), even in ETFR cells, which express high levels of FPR. Generally, an active CaG peptide, or an active variant thereof, that mimics CaG will selectively activate PKC ζ, but will not activate PKC α or PKC β. Methods to test PKC activation are conventional. Some such methods are described in the Examples.

With regard to the activation of calcium flux: In general, CaG does not induce substantial amounts of calcium flux in FPR expressing cells. When monocytes or neutrophils are treated with CaG, the degree of calcium flux is induced to very low levels, even with calcium concentrations up to 20 μg/ml (800 nM). But when other cells, such as ETFR cells, are tested, weak amounts of calcium flux can be observed. This effect presumably results from the fact that the cells express considerably more FPR than do the non-transfected, cell types. It will be evident to the skilled worker how much induction of calcium flux in a given cell type qualifies as being an absence of substantial induction of calcium flux. In general, CaG, or an active peptide of CaG, induces substantially less calcium flux than does FMLP (formyl-methionyl-leucyl-phyenyalanine). See the Examples herein.

With regard to the activation of mitogen-activated protein kinases (MAP kinases): In general, active CaG peptides, mimetics thereof, and improved agonists of the invention, induce substantial activation (e.g., phosphorylation) of MAP kinases (members of an intracellular signaling cascade, including but not limited to the MAP kinases whose activation is illustrated in the Examples), although CaG appears to be a less effective ligand in this respect than FMLP. See the Examples herein.

One aspect of the invention is a method for identifying an agent (e.g., a small molecule) that modulates the agonist ability of CaG on FPR, comprising screening a candidate agent for its ability, when contacted with an FPR-expressing cell, in the presence of CaG, to enhance the agonist activity, e.g., to:
a) activate PKC ζ, PKC α and/or PKC β to a greater degree than does CaG; and/or
b) induce a greater amount of calcium flux than does CaG; and/or
c) induce a greater amount of chemotaxis of the cell than does CaG; and/or
d) activate a MAP kinase to a greater degree than does CaG;
or to inhibit the agonist activity (to act as an antagonist), e.g., to
a) activate PKC ζ, PKC α and/or PKC β to a lesser degree than does CaG; and/or
b) induce a lesser amount of calcium flux than does CaG; and/or
c) induce a lesser amount of chemotaxis of the cell than does CaG; and/or
d) activate a MAP kinase to a lesser degree than does CaG.

A variety of classes of putative stimulatory (or inhibitory) agents (compounds) can be tested by such screening methods. Preferably, the method is used to test small molecule compounds, e.g., compounds from a combinatorial library.

“Small molecules,” sometimes referred to herein as “compounds,” can be generated as follows: Such small molecules may be isolated from natural sources or developed synthetically, e.g., by combinatorial chemistry. In general, such molecules are identified from large libraries of natural products or synthetic (or semi-synthetic) extracts or chemical libraries according to methods known in the art. Those skilled in the field of drug discovery and development, for example, will understand that the precise source of test extracts or compounds is not critical to the methods of the invention. Accordingly, virtually any number of chemical extracts or compounds can be used in the methods described herein. Examples of such extracts or compounds include, but are not limited to, plant-, fungal-, prokaryotic- or animal-based extracts, fermentation broths, and synthetic compounds, as well as modification of existing compounds. In particular, it may be desirable to start with a known small molecule agonist of FPR or CaG and to modify it to optimize its stimulatory properties and/or to reduce its toxic properties; or to modify an FPR or CaG antagonist, in order to convert it into an FPR agonist (or vice-versa). Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.).

Alternatively, libraries of natural compounds in the form of bacterial, fungal, plant, and animal extracts are commercially available from a number of sources, e.g., Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

The screening methods of the invention are generally carried out in vitro (e.g., ex vivo). That is, the FPR-expressing cell is generally contacted with the candidate (e.g., peptide) in a test tube or other device, or in culture. Alternatively, the method may be carried out in vivo. For example, the cell may be contacted with the candidate in, or may be implanted into, an animal, e.g., a mammal, such as a laboratory animal (e.g., mouse, rat, guinea pig, rabbit, etc.), cat, dog, horse, non-human primate, or a human In one embodiment, the ability of a molecule to induce cell recruitment is tested in mice, e.g., by s.c. (subcutaneous) or intraperitoneal injection.

The order and numbering of the steps in the methods described herein are not meant to imply that the steps of any method described herein must be performed in the order in which the steps are listed or in the order in which the steps are numbered. The steps of any method disclosed herein can be performed in any order which results in a functional method. Furthermore, the method may be performed with fewer than all of the steps, e.g., with just one step.

In the assays described herein, a putative agent (e.g., a CaG-related peptide) may or may not modulate a desired CaG-FPR induced response (e.g., partial agonist response). In a general sense, this invention relates to methods to determine if a putative agent exhibits such modulatory behavior, irrespective of whether such modulation is detected.

Screening methods of the invention can be adapted to any of a variety of high throughput methodologies. High throughput assays are generally performed on a large number of samples, and at least some of the steps are performed automatically, e.g., robotically.

A CaG-related peptide of the invention that mimics CaG (a peptide mimetic), as used herein, is a peptide of CaG, or an active variant thereof, which exhibits at least one CaG-FPR mediated activity. Preferably, the mimetic exhibits all of the activities exhibited by CaG.

An agent of the invention that acts as an FPR agonist (either a CaG-related peptide or a small molecule agonist) is an agent that induces (enhances, promotes, activates, increases, etc.) at least one CaG-FPR mediated activity. Peptide agonists of the invention include CaG peptides (from a wild type CaG) that elicit stronger agonist activity than does the full-length protein; or peptides which are active variants of a CaG peptide.

A CaG-related peptide of the invention which acts as an antagonist of FPR includes a peptide that binds to FPR but does not stimulate an FPR activity (e.g., is a competitive inhibitor for the binding of CaG to FPR). Without wishing to be bound by any particular mechanism, it is suggested that the inventive antagonists of FPR can inhibit the interaction between FPR and CaG, for example by binding to the receptor or to the ligand, and/or by blocking access of FPR by its agonists through steric hindrance on the cell membrane.

Another aspect of the invention is a kit, suitable for performing any of the methods of the invention. The components of the kit will vary according to which method is being performed. In general, a kit of the invention comprises a suitable FPR-expressing cell and means (e.g., suitable reagents) for detecting one or more elements of a CaG/FPR-induced response. Reagents for performing suitable controls may also be included.

Optionally, the kits comprise instructions for performing the method. Kits of the invention may further comprise a support on which a cell can be propagated (e.g., a tissue culture vessel) or a support to which a reagent used in the method is immobilized. Other optional elements of a kit of the invention include suitable buffers, media components, or the like; a computer or computer-readable medium for storing and/or evaluating assay results; logical instructions for practicing the methods described herein; logical instructions for analyzing and/or evaluating assay results as generated by the methods herein; containers; or packaging materials. The reagents of the kit may be in containers in which the reagents are stable, e.g., in lyophilized form or stabilized liquids.

A modified agent of interest (e.g., a CaG peptide or an active variant thereof) can be characterized by performing assays (e.g., with a kit of the invention), and comparing the results to those obtained with the unmodified agent (e.g., full length CaG or a CaG peptide, respectively), or by comparison to a reference database.

The kits and methods of the invention have many uses, which will be evident to the skilled worker. For example, they can be used in experiments to study the biological role of CaG in host response in inflammation, immunity, and microbial infection; and to identify CaG-related peptides that act as mimics, agonists or antagonists as described herein.

Peptides identified by methods of the invention may mediate any of a variety of cellular or physiological responses. These activities may also be screened for when identifying CaG-related peptides of the invention. Peptides identified by methods of the invention may be used in a variety of methods to induce or activate (or, in the case of antagonists, to inhibit) any of these activities.

For example, FPR is thought to play a significant role in host defense against microbial infection by promoting phagocyte recruitment and secretion of bactericidal mediators. Evidence to support this assumption includes a report of reduced resistance to Listeria monocytogenes infection by mice devoid of mFPR1, a mouse orthologue of human FPR. Furthermore, in humans, it has been reported that dysfunctional variant FPR alleles are associated with localized juvenile periodonitis caused by Actinobacillus actinomycetescomitans; and the FPR variants encoded by mutated genes exhibited severe or complete deficiency in Gi protein coupling in transfected cells, consistent with a direct causal role of dysfunctional FPR in the disease. Thus, both human and mouse models of diseases imply active participation of FPR in anti-microbial responses. Moreover, CaG-related agonists of antagonists of FPR would be expected to inhibit virus infection as well. For example, an FPR agonist would be expected to inhibit HIV by activating FPR and thereby desensitizing HIV co-receptors.

Also, FPR has been reported to participate in the fine-tuning of neutrophil extravasation when multiple chemoattractants are present, and antagonists of FPR have been reported to negatively regulate the inflammatory response. The release of chemoattractant peptides (such as CaG) by neutrophils has been postulated as an important step in the chain of events galvanizing host immune system during inflammation, infection and wound healing. Since FPR is expressed by a variety of cell types including immature dendritic cells, CaG may also act as a link between innate and adaptive immunity by recruiting DC, and CaG-FPR interaction may modulate the function of non-hematopoietic cells that express functional FPR.

Other cellular activities mediated by CaG and FPR are discussed elsewhere herein, e.g., in relation to CaG/FPR induced responses.

The invention includes CaG-like peptides identified by a method of the invention (peptides of the invention), and methods of using such peptides. Thus, other embodiments of the invention include:

A method for treating a subject in need of such treatment (e.g., a human patient or other animal subject having a condition or disease that is mediated by an aberrantly high or low amount of a CaG/FPR-induced activity), comprising administering to the subject an effective amount of a peptide of the invention. Such a method can, e.g., treat, prevent, ameliorate, control, suppress, stop, slow and/or inhibit the condition.

A pharmaceutical composition, which comprises a peptide of the invention (e.g., a therapeutically effective amount of the peptide) and a pharmaceutically acceptable carrier.

A method for stimulating a CaG-mediated process in a cell that expresses FPR, comprising contacting the cell with an effective amount of a CaG mimetic or FPR agonist of the invention.

A method for inhibiting a CaG-mediated process in a cell that expresses FPR, comprising contacting the cell with an effective amount of an FPR antagonist of the invention. An inhibitory peptide (an FPR antagonist) can be used in conjunction with one or more known FPR inhibitors, such as the gall bladder products deoxyxholic acid (DCA) or chenodeosycholic acid (CDCA), or the bacterial products cyclosporine A or cyclosporine H; a competitive inhibitor (e.g., a peptide) of FPR (such as Boc-FLFLF (SEQ ID NO: 2)); a recombinant construct that expresses a peptide competitive inhibitor; or an antibody specific for FPR.

Another aspect of the invention is an isolated complex of CaG and FPR. By an “isolated” complex is meant herein one that is in a form not found in its original environment or in nature, e.g., more concentrated, more purified, separated from at least one other component with which it is naturally associated, in a buffer, etc. The “complex” includes CaG and FPR molecules that are associated in any form, e.g., covalently, non-covalently, etc. In embodiments of the invention, the complex is in a membrane or is in a cell (which can be any cell comprising FPR, such as a neutrophil, etc.); and/or the complex is in vitro.

In the foregoing and in the following examples, all temperatures are set forth in uncorrected degrees Celsius; and, unless otherwise indicated, all parts and percentages are by weight.

### EXAMPLES

### Example I

### Materials and Methods

A. Reagents and cells

Cathepsin G, purified from neutrophil granules, was purchased from Athens Research & Technology (Athens, Ga.). The synthetic N-formyl peptide fMLF, α1-antichymotrypsin (α1-ACT), PMA, Staurosporine, and Wortmannin were purchased from Sigma (St. Louis, Mo.). MMK-1 peptide was synthesized and purified by the Department of Biochemistry, Colorado State University (Fort Collins, Colo.) according to the published sequence (Klein et al. (1998) Nat Biotechnol 16, 133411). Gö6850, and chelerythrine chloride were from CalBiochem (La Jolla, Calif.). Fura-2/AM was obtained from Molecular Probes (Eugene, Oreg.). Anti-human FPR antibody was from PharMingen (San Diego, Calif.). Cyclosporin H (CsH) was a kind gift from Novartis (Basel, Switzerland). Anti-total- and phosphorylated (p)-ERK1/2, p38 MAPK, and Akt antibodies were products of Cell Signaling (Beverly, Mass.). Anti-PKCα/β, δ, and ξ, polyclonal antibodies were obtained from Santa Cruz Biotechnology (Santa Cruz, Calif.).

Human PBMC were isolated from leukopacks obtained through the courtesy of Transfusion Medicine Department, National Institutes of Health Clinical Center (Bethesda, Md.) by Ficoll-Hypaque density gradient centrifugation. Monocytes were purified from human PBMC by Percoll gradient to yield>90% pure preparations. Rat basophilic leukemia cells (RBL cells) stably transfected with epitope-tagged high affinity fMLF receptor FPR (designated ETFR cells) were a kind gift of Drs. H. Ali and R. Snyderman, Duke University (Durham, N.C.) and maintained in the presence of 0.8 mg/ml geneticin (G418, Life Technologies, Rockville, Md.) in DMEM supplemented with 10% FCS. B. Chemotaxis assay Migration of phagocytes and ETFR cells was assessed using a 48-well microchemotaxis chamber (Neuroprobe, Cabin John, Md.) as previously described (Hu et al. (2001) J Leukoc Biol 70 15512). Briefly, different concentrations of stimulants were placed in the wells of the lower compartment of the chamber, and 50 μl cells (1.5×10⁶ cells/ml for monocyte and neutrophil, 1×10⁶ ETFR cells/ml) were added to the wells of the upper compartment. The lower and upper compartments were separated by either a 5-μm (for monocyte) or a 10-μm pore size, collagen-coated (for ETFR cells) polycarbonate filter (Osmonics, Livermore, Calif.). After incubation at 37° C. in humidified air with 5% CO₂ (60 min for neutrophils, 90 min for monocytes, 4.5 h for ETFR cells), the filter was removed and stained, and the cells migrating across the filter were counted with light microscopy. The results are presented as the mean number of cells (±SD) in three high power fields (HPFs). For analysis of cross-desensitization, cells were pretreated with indicated reagents for 1 h at 37° C. After thorough washing with cold medium (RPMI 1640, 1% BSA), the cells were resuspended and assayed. To measure the effect of various signaling inhibitors, unless otherwise indicated, the cells were pre-incubated at 37° C. for 60 min with the inhibitors, and were then added to the upper wells of the chemotaxis chamber.

C. Calcium Flux

Monocytes or ETFR cells (10⁷ cells/ml in RPMI 1640 containing 10% FBS) were loaded with 5 μM Fura-2 at room temperature (30 min for monocytes, 60 min for ETFR cells) in the dark. The cells were washed and resuspended (0.5×10⁶ cells/ml) in saline buffer (138 mM NaCl, 6 mM KCl, 1 mM CaCl₂, 10 mM HEPES, 5 mM glucose, and 1% BSA, pH 7.4). Two milliliters of the cell suspension were transferred to a quartz cuvette, which was placed in a luminescence spectrometer LS55 (Perkin-Elmer, Beaconsfield, UK). Calcium mobilization was measured by recording the ratio of the fluorescence excited at 340 and 380 nm in response to stimulants.

D. Phosphorylation of Mitogen Activated Protein Kinases (MAPKs) and Akt

ETFR cells were plated in Petri dishes and cultured in DMEM at 37° C. in humidified 5% CO₂ atmosphere for 48 h. After further culture in serum-free DMEM overnight, the cells were treated with stimulants for indicated time periods. The reaction was stopped by adding 1 ml ice-cold PBS, and the cells were lysed in 1×SDS lysis buffer (62.5 mM Tris-HCl, 2% w/v SDS, 10% glycerol, 50 mM DTT, pH 6.8) and harvested into tubes for sonication. Proteins (50 μg for each sample) were resolved by SDS-PAGE and transferred onto nitrocellulose membranes (Bio-Rad, Hercules, Calif.). The membranes were blocked for 2 h at room temperature in 3% non-fat milk prepared in Tris-buffered saline-T (10 mM Tris-HCl, pH 7.5, 150 mM NaCl, 0.1% Tween 20) and probed with anti-p-ERK1/2, p-p38 MAPK, or p-Akt (1:1000) at room temperature for 1-2 h or at 4° C. overnight. The membranes were then washed and incubated with horseradish peroxidase-conjugated secondary antibodies diluted in Tris-buffered saline-T for 1 h at room temperature. The immune complexes on the membranes were visualized using ECL reagents. Total ERK1/2, p38 MAPK, and Akt were visualized by stripping the same membrane and probing with antibodies against total ERK1/2, p38 MAPK, and Akt.

E. Measurement of FPR Cell Surface Expression

Cell surface expression of FPR was monitored by a flow cytometer (FACS). Monocytes and ETFR cells (1×10⁶ cells/sample) were pretreated with medium or stimulants at 37° C. for 30 minutes. After two washes with cold FACS buffer (DPBS, 1% FCS, 0.02% sodium azide, and 5 mM EDTA, pH7.4), the cells were blocked with 5% normal rat serum followed by incubation with a monoclonal anti-human FPR antibody or isotype matched IgG on ice for 30 min. After three washes with FACS buffer, FITC-labeled rat anti-mouse antibody (PharMingen) was applied and incubated on ice for 30 min. The cells were then washed twice with FACS buffer and analyzed immediately by flow cytometry (Becton Dickinson, San Jose, Calif.).

F. Binding Assays with ³H-fMLF and ¹²⁵I-CaG

³[H]-fMLF was purchased from Amersham Biosciences (Piscastaway, N.J.). Human monocytes or ETFR cells (2×10⁶ cells in 100 μl RPMI160 containing 1% BSA and 0.5% sodium azide) in duplicate samples were incubated with 3 nM of ³[H]-fMLF in the presence of increasing concentrations of unlabeled fMLF or CaG at room temperature for 30 min. The cells were then filtered onto Whatman paper discs, which were air-dried and measured for Remission. The level of non-specific ³[H]-fMLF binding to the cells was determined by the residual radioactivity on cells in the presence of 10 μM unlabeled fMLF (<20 of total binding in the absence of unlabeled fMLF). The results were presented as the capacity of CaG to displace specific ³[H]-fMLF binding to the cells (% inhibition).

CaG was iodinated with a chrolamine-T method (Lofstrand Labs, Gaithersburg, Md.) with a specific activity of 15.8 pCi/μg protein. Duplicate samples of ETFR cells or the parental RBL cells (2×10⁶ cells in 200 μl RPMI 1640 containing 1% bovine serum albumin, 25 mM Hepes, and 0.05% NaN₃) were incubated with 25 nM ¹²⁵I-CaG, in the presence of increasing concentrations of unlabeled ligands. After incubation and rotation at room temperature for 1 h, the cells were pelleted through a 10% sucrose/PBS cushion for 1 min at 10,000 g. The supernatant was removed and the radioactivity associated with cell pellets was measured in a γ counter (CliniGamma, Pharmacia Biotech Inc.). The binding data were analyzed with a Macintosh computer-aided program LIGAND (Dr. P. Munson, Division of Computer Research and Technology, NIH, Bethesda, Md.).

G. Measurement of Protein Kinase C (PKC) Translocation and FPR Internalization

ETFR cells were seeded on coverslips and cultured overnight in DMEM containing 1% FCS at 37° C. After further culture in FCS-free DMEM for 3 h, cells were treated with stimulants for indicated time periods at 37° C. and were then fixed in 4% paraformaldehyde for 5 min at room temperature. The cells were permeabilized with 0.2% Tween 20 for 30 min at 4° C. and the reaction was blocked with 5% goat normal serum for 15 min at room temperature. The rabbit anti-PKCα/β, δ, and ξ, antibodies (1:1000 in DPBS) were applied separately for 1 h at room temperature. After three rinses with DPBS, the cells were stained with FITC-labeled goat anti-rabbit secondary antibody (PharMingen, 1:1000 in DPBS) for 1 h at room temperature. The coverslips were mounted with an anti-fade, water-based mounting medium with Propidium Iodide (PI) (Vector Lab, Burlingamem, Calif.) and analyzed under a laser scanning confocal fluorescence microscope (Zeiss LSM510 NLO Meta, Jena, Germany). Excitation wavelengths 488 (for FITC) and 568 (for PI) nm were used to generate fluorescence emission in green and red, respectively.

For detection of FPR internalization, ETFR cells were seeded on coverslips and cultured overnight in DMEM containing 10% FCS at 37° C. After pretreatment with medium or stimulants for 30 minutes at 37° C., the cells were fixed in 0.25% paraformaldehyde for 15 min at 4° C. The cells were then permeabilized with 0.2% Tween 20 for 30 min at 4° C., followed by incubation with a monoclonal anti-human FPR antibody or isotype matched IgG for 30 min at 4° C. After three washes with 0.1% Tween 20 in DPBS, a FITC-labeled rat anti-mouse antibody (PharMingen) was applied to the cells for 30 min at 4° C. The coverslips were covered with DAPI, then mounted with an anti-fade, water-based mounting medium and analyzed under a laser scanning Zeiss LSM410 fluorescence confocal microscope.

H. Statistical Analysis

All results shown were representatives from at least 3 experiments. The statistical significance of the differences in cell migration between testing and control groups was analyzed using a Student's t test.

### Example II

### Cathepsin G Induces Phagocyte Migration Mediated by Formyl Receptor FPR

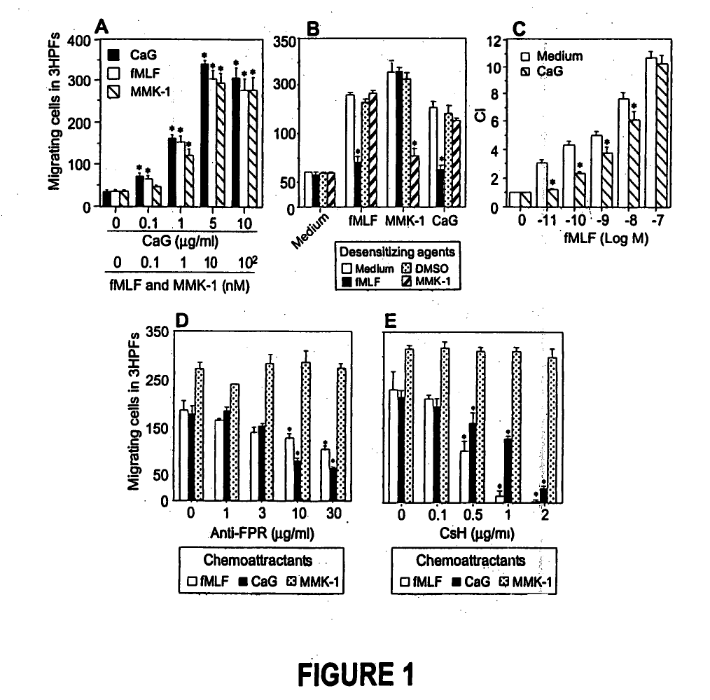

In order to define the nature of the putative receptor(s) used by CaG on phagocytes, we examined the capacity of a number of chemoattractants to desensitize phagocyte response to CaG, an approach used previously to identify shared or unique G protein coupled STM receptors. Although cross-desensitization of Ca²⁺ mobilization is a reliable and commonly used strategy, CaG at the concentrations up to 20 μg/ml (800 nM) lacked significant Ca²⁺ mobilizing activity in either monocytes or neutrophils. Therefore, we tested cross-attenuation of chemotaxis. CaG dose-dependently induced monocyte migration (FIG. 1A), which reached maximal activity at a concentration of 5 μg/ml of the protein (200 nM). Of the many chemoattractants tested, the bacterial chemotactic peptide fMLF attenuated monocyte response to CaG (FIG. 1B). Since fMLF has been reported to activate at least two human formyl peptide receptors, the high affinity receptor FPR and the low affinity FPRL1, we tested the capacity of agonists specific for these two receptors to attenuate the monocyte response to CaG. MMK-1, known to be a specific and highly efficacious peptide agonist for FPRL1 (FIG. 1A), did not affect CaG-induced monocyte chemotaxis (FIG. 1B). On the other hand, fMLF at nanomolar concentrations effectively attenuated monocyte migration induced by CaG. CaG was also able to attenuate monocyte chemotaxis to low concentrations of fMLF but not MMK-1 (FIG. 1C). Since at low concentrations, fMLF has been shown to specifically activate FPR, we postulated that CaG shared FPR with fMLF for its phagocyte chemotactic activity. This hypothesis was tested with a monoclonal antibody against FPR, which when preincubated with monocytes, inhibited cell migration to either CaG or fMLF, but not to the FPRL1 agonist MMK-1 (FIG. 1D). In addition, cyclosporin H (CsH), an antagonist known to specifically disrupt FPR signaling and inhibit fMLF-induced phagocyte activation, abolished monocyte chemotaxis to CaG and fMLF but not to MMK-1 (FIG. 1E). Thus, CaG appears to use FPR to induce chemotaxis of monocytes.

### Example III

### Cathepsin G Induces Chemotaxis of RBL Cells Stably Transfected with Human FPR

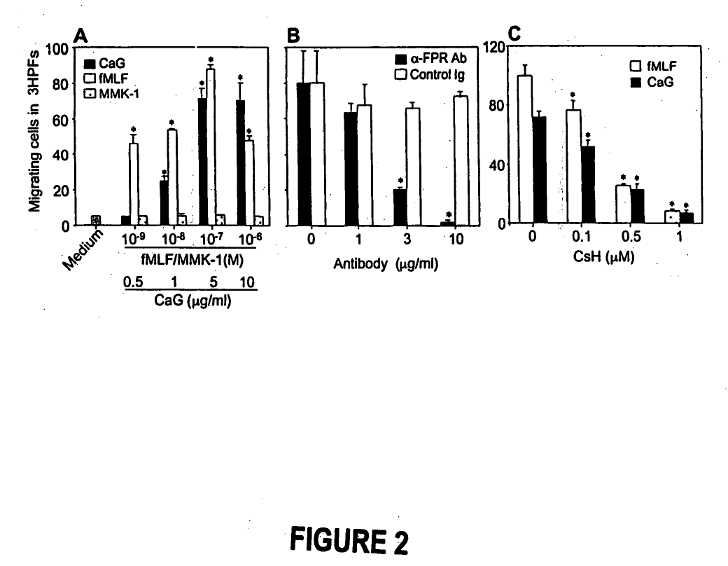

To confirm that CaG uses FPR, we used RBL cells transfected to solely express this receptor (ETFR cells). ETFR cells showed a marked and dose-dependent chemotactic response to CaG and fMLF (FIG. 2A). The same cells failed to respond chemotactically to MMK-1, a ligand for FPRL1. Similar to its activity on monocytes, the optimal concentration for CaG to induce ETFR cell migration was 5 μg/ml. Neither CaG nor fMLF induced migration of parental RBL cells suggesting the presence of FPR is required for CaG- and fMLF-induced chemotaxis. Furthermore, anti-FPR antibody (FIG. 2B) and CsH (FIG. 2C) abolished ETFR cell response to CaG. These results confirm CaG as a chemotactic ligand for FPR.

### Example IV

### Characteristics of CaG-Induced FPR Signaling

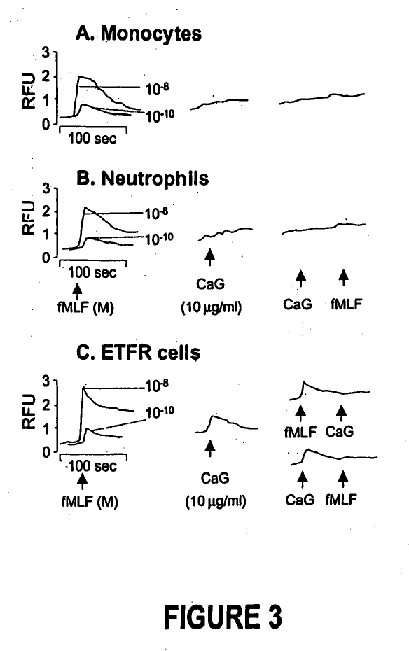

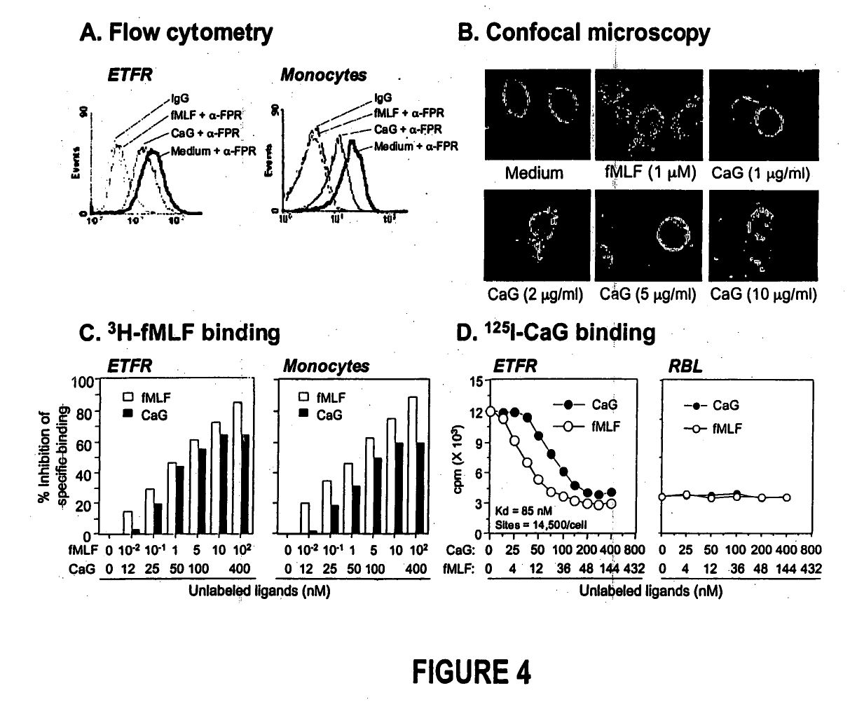

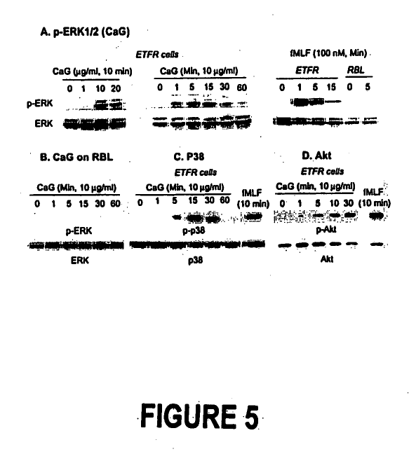

The signaling events induced by CaG through FPR exhibited several unique characteristics. Although CaG at low micrograms/ml (in the 4-400 nM range) was potently chemotactic for monocytes, neutrophils and RBL cells transfected to express FPR (ETFR cells), it was a weak inducer of Ca²⁺ flux only in ETFR cells (FIGS. 3A, B, C), presumably due to an markedly higher level expression of transfected FPR. Pretreatment of the cells with 10 μg/ml CaG attenuated cell response induced by low concentrations of fMLF (FIGS. 3A, B, C), suggesting that CaG exhibits lower affinity for FPR as compared to fMLF. This notion was supported by a weaker, but significant ability of CaG to down-regulate FPR from the surface of ETFR cells and monocytes (FIG. 4A) as compared to a marked effect of fMLF. The down-regulation of FPR from the cell surface induced by CaG was associated with receptor internalization as assessed by confocal microscopy. FIG. 4B shows that FPR expressed on ETFR cells was rapidly internalized into the cytoplasmic region of the cells upon treatment with fMLF. CaG, albeit exhibiting a lower efficacy than fMLF, also significantly and dose-dependently induced FPR internalization. In receptor binding competition experiments, unlabeled fMLF displaced ³[H]-fMLF binding to monocytes and ETFR cells with an IC 50 of about 3 nM, whereas CaG exhibited an IC 50 of 100 nM (2.5 μg/ml) (FIG. 4C). ¹²⁵I-CaG was used to directly measure its capacity to bind to RBL cells with or without FPR. ETFR cells exhibited a substantial number of binding sites for ¹²⁵I-CaG with an estimated Kd of 85 nM (approximately 2 μg/ml) (FIG. 4D). The binding of ¹²⁵I-CaG to ETFR cells was potently inhibited by fMLF (FIG. 4D(1)). In contrast, there was no measurable levels of specific binding of ¹²⁵I-CaG to parental RBL cells (FIG. 4D(2)). These results provide additional supporting evidence that CaG directly interacts with FPR. CaG also positively and rapidly (within 1-5 min) activated ERK1/2 (FIG. 5 A), p38 MAPK (FIG. 5 C), and Akt (FIG. 5 D), molecules downstream of the FPR signaling cascade in ETFR cells in both a time- and dose-dependent manner. The parental RBL cells did not show any increased phosphorylation of ERK1/2 in response to CaG (FIG. 5B). In parallel experiments to measure p-ERK, fMLF at 100 nM showed a maximal effect at 1 min (FIG. 5A). Although these results suggest that compared to fMLF, CaG appears to be a less efficacious ligand for FPR, nevertheless whether a slightly slower rate of CaG in inducing p-ERK in ETFR cells implies the generation of new FPR agonist(s) by this neutrophil protein merits further investigation.

Despite its apparently lower affinity for FPR as compared to fMLF, CaG may be one of the most potent chemotactic factors released by neutrophils during degranulation, and micromolar concentrations of CaG could potentially be secreted at sites of acute inflammation and infection where large quantity of neutrophils accumulate. The low affinity agonist-receptor interaction may contribute to the recruitment of leukocytes to sites where high concentrations of the chemoattractant ligands are generated.

### Example V

### FPR-Dependent Activation of PKCξ by CaG

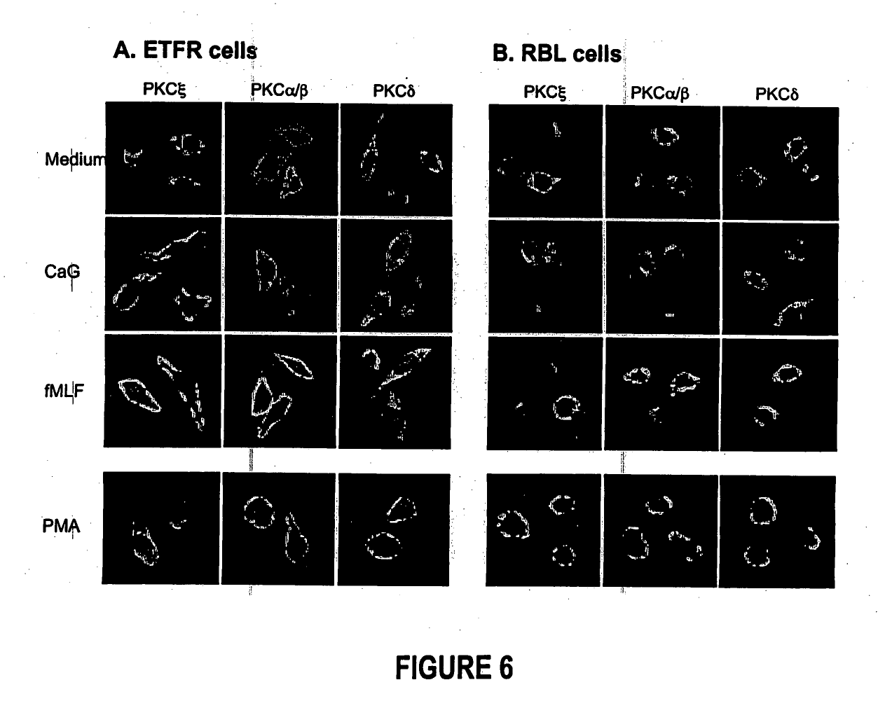

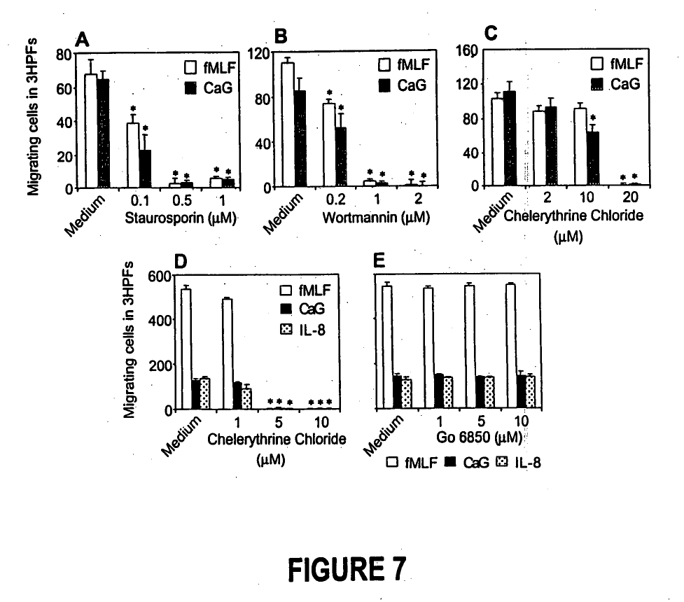

Translocation of PKC isozymes to different cellular compartments is a hallmark of selective PKC activation (Mochly-Rosen et al. (1995) Science 268, 247). Among the PKC isozymes, PKCξ has been reported as a critical signal transducer in promoting cell migration induced by a variety of stimulants (Etienne-Manneville et al. (2001) Cell 106, 489; Laudanna et al. (1998) J Biol Chem 273, 30306). In unstimulated ETFR cells, PKCξ was mainly detected in the cytoplasm. Following stimulation with CaG, a substantial proportion of PKCξ translocated to the cell membrane region as assessed by confocal microscopy (FIG. 6A). The induction of PKCξ translocation by CaG was dependent on the presence of FPR because CaG did not cause any changes in the pattern of PKCξ distribution in the parental RBL cells (FIG. 6B). CaG did not induce translocation of PKCα and β in ETFR cells, two members of the classical PKC subfamily which are dependent on the generation of diacyl glycerol (DAG) and Ca²⁺, two second messengers coupled to many cell surface receptors. This is agreement with a weaker capacity for CaG to trigger FPR-dependent Ca²⁺ mobilization. In contrast, fMLF, which elicits both FPR-dependent chemotaxis and Ca²⁺ flux, promoted the translocation of not only PKCξ, but also PKCα/β (FIG. 6A). Neither CaG nor fMLF stimulated PKC translocation in parental RBL cells, while PMA activated these PKC isozymes in both ETFR and RBL cells (FIGS. 6A, B). CaG and fMLF did not induce translocation of PKCδ, a member of the novel PKC subfamily, suggesting that activation of this PKC isozyme may not be involved in the signaling of FPR upon ligation with CaG or fMLF. Furthermore, both Staurosporine, a general PKC inhibitor, and Wortmannin, a PI3K inhibitor, abrogated chemotaxis of ETFR cells induced by either CaG or fMLF (FIGS. 7A, B), and chelerythrine chloride, a specific PKCξinhibitor markedly inhibited the cell migration to CaG and fMLF (FIG. 7C). In contrast, Gö 6850 (bisindolylmaleimide I), an inhibitor of classical (α, β₁ and β₂) and novel (δ, ε, η, and θ) PKC had a limited effect on fMLF- and CaG-induced ETFR cell migration. These results were also confirmed in human neutrophils where the PKCξ inhibitor chelerythrine chloride abolished the cell chemotaxis to CaG, fMLF as well as a chemokine IL-8, while Gö 6850 did not have any effect (FIGS. 7D, E).

### Example VI

### CaG does not Generate New and Soluble FPR Agonist(s)

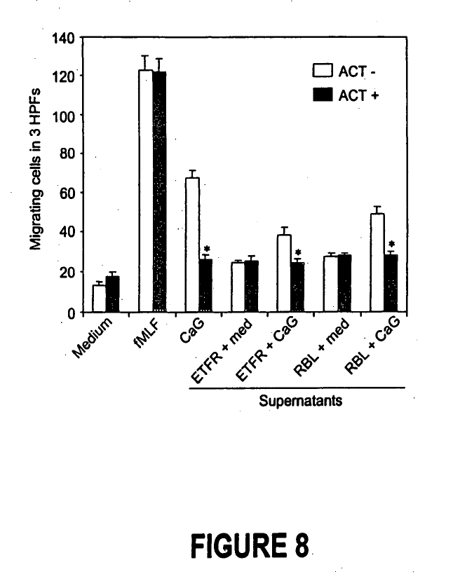

Cleavage by proteases is required for regulation of the function of a number of molecules in triggering proper physiological processes. Modification of the enzymatic site of CaG with PMSF and incubation with α1-ACT has been shown to inhibit its chemotactic activity, implying that an enzymatically intact form of CaG is important for its phagocyte chemotactic activity. Therefore, we examined whether interaction of FPR with CaG might cleave FPR to generate a new defacto agonist for this receptor, such as in the model of thrombin activation of its receptors. ETFR cells were cultured with CaG under the conditions that are sufficient for cleavage of other proteins by CaG (Kinlough-Rathbone et al. (1999) Thromb Res 95, 31524). The supernatant was collected and examined for its chemotactic activity for FPR. FIG. 8 shows that although the supernatant from CaG-digested ETFR cells contained a low level of chemotactic activity, such activity was completely abolished by additional treatment with the CaG inhibitor α1-ACT, suggesting that the chemotactic activity contained in the supernatant of CaG-digested ETFR cells is due to the residual active CaG but not a newly generated FPR agonist. Supernatants from CaG-treated RBL cells also exhibited some chemotactic activity for ETFR cells, which was inhibited by α1-ACT, suggesting that CaG also did not generate any new FPR agonists from RBL cells (FIG. 8). α1-ACT did not affect the chemotactic activity of fMLF for FPR (FIG. 8), supporting the notion that CaG is unlikely to cause the release of small and soluble peptide agonist(s) during its interaction with FPR-expressing cells. Also, detection of CaG-induced FPR internalization as shown in FIG. 4B by confocal microscopy and direct binding of ¹²⁵I-CaG to ETFR cells argues against the potential cleavage of FPR by CaG. Nevertheless, whether CaG may generate chemotactic molecules that could tightly associate with the membrane on the leading edge of migrating cells remains to be clarified.

### Example VII

### Generating and Testing CaG Peptides

Peptides are synthesized by Fmoc chemistry on a peptide synthesizer and purified by RP-HPLC on a Resource RPC column. The sequences of the synthetic peptides are confirmed by Edman degradation on a 477A/120A protein sequencer.

An overlapping series of peptides of about 15-25 (preferably about 20) amino acids is prepared corresponding to the central portion (about the central third) of the CaG protein (about 80 amino acids). For example, 20 amino acid peptides extending from amino acid 85-104, 88-107, etc. through the central portion of the protein are generated. Each of these peptides is contacted with a humancyte, neutrophil, or cell line transfected with FPR and tested, e.g., for its ability to modulate a CaG/FPR activity, such as activation of PKC ζ, PCKαor PKC β in the cell; induction of calcium flux; induction of chemotaxis; and/or the ability to activate a MAP kinase. Not all candidate peptides of CaG would be expected to modulate a CaG/FPR response. Using this type of assay, the inventors identify a subset of CaG peptides that, unexpectedly, do exhibit such activity.

Peptides which exhibit desirable properties (e.g., mimic a CaG/FPR activity, act as a more potent agonist than CaG, or act as an antagonist of CaG) are then modified, in order to optimize the effect. For example, one, two, three or more amino acids are added to one or both ends of the peptide. The added sequences are generally chosen from the sequence of the full-length CaG protein, although variants of those sequences may also be added. Alternatively, mutations are made within the 20-mer sequences. For example, amino acid substitutions (preferably conservative substitutions) are made at either or both of the N- and C-termini of a peptide. The modified peptides are tested as above, and suitable peptides are selected.

A similar series is overlapping peptides is generated from the N-terminal third of CaG, and a similar series is generated from the C-terminal third of CaG. These peptides are tested, modified, and retested as is done for the internally located peptides.

For the most promising peptides, animal experiments are carried out, using conventional procedures. For example, the ability of the peptides to induce cell recruitment is tested in mice, by s.c. (subcutaneous) or intraperitoneal injection.

These experiments allow the identification of domains within CaG that are responsible for the various CaG/FPR activities discussed herein. They also allow for the identification of agonists or antagonists of FPR for the various activities.

### Example VIII

### The N-Terminus of FPR is not Required for the Chemotactic Activity of CaG

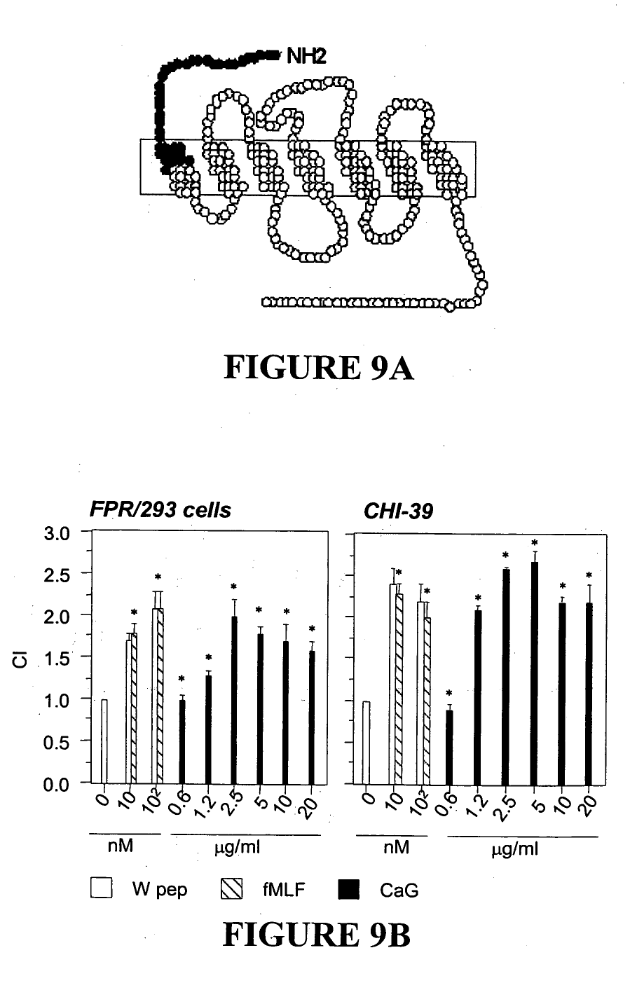

To identify functional domains of the FPR receptor for chemotactic activity of CaG, a chimeric receptor, called CHI-39, was generated. This chimeric receptor contains the FPR backbone; however, the N-terminal sequences are substituted with the comparable N-termina; sequence from human FPRL1 (formyl peptide receptor-like 1), a variant of FPR. A schematic representation of CHI-39 is shown in FIG. 9A.

CHI-39 was generated by the methods described in Le et al. (2005) FEBS Journal 272, 769-778 and Quehenberger et al. (1993) J Biol Chem 268, 18167-18175, except in the present example FPR sequences are substituted with the corresponding amino acid sequences from FPRL1, whereas in the reference FPRL1 sequences are substituted with corresponding amino acid sequences from FPR.

The chimeric receptor was stably transfected into HEK293 cells (a human embryo kidney cell line) and the chemotactic activity of CaG on the transfected cells was measured and compared to the chemotactic activity of CaG on HEK39 cells transfected with wild type FPR, using the assay for chemotaxis described in Example IB. FIG. 9B shows the chemotactic responses to chemoattractants W peptide (W pep), fMLF and CaG. W peptide and fMLF, at the indicated concentrations, were used as controls. The results are presented as chemotaxis index (CI) defining the fold increase of migrating cells in response to receptor agonists over cell migration in the absence of agonists. * denotes statistically a significant fold increase (P<0.01). FIG. 9B shows that cathepsin G is chemotacic for FPR-transfected HEK 293 cells (left side of figure) and that, when the N-terminus of FPR is replaced by comparable sequences from FPRL1 (a receptor that is not responsive to CaG), the transfected HEK293 cells nevertheless exhibit robust migration in response to CaG (right side of figure). Thus, the N-terminal sequences of FPR are not required for the chemotactic activity of CaG.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and below and in the figures are hereby incorporated by reference.

We claim:

1. A method for identifying a peptide of cathepsin G (CaG), or a peptide which is an active variant thereof, that modulates a CaG-FPR induced activity, comprising contacting a candidate peptide with a cell comprising an FPR receptor, and determining the effect of the peptide on the receptor.

2. The method of claim 1, wherein the candidate peptide is screened for its ability, when contacted with an FPR-expressing cell, to activate PKC ζ, PKC α and/or PKC β, in the cell.

3. The method of claim 1, wherein the candidate peptide is screened for its ability to induce calcium flux.

4. The method of claim 1, wherein the candidate peptide is screened for its ability to induce chemotaxis of the cell.

5. The method of claim 1, wherein the candidate peptide is screened for its ability to activate a MAP kinase.

6. The method of claim 1, which is a method to identify a peptide that mimics CaG, wherein a candidate peptide that is shown to selectively activate PKC ζ, but not PKC α and/or PKC β, in the cell; and/or not to induce substantial amounts of calcium flux, is a peptide that mimics CaG.

7. The method of claim 1, which is a method to identify a peptide that is a more potent agonist of FPR than is CaG,
wherein a candidate peptide which, when contacted with an FPR-expressing cell is shown to
a) activate PKC ζ, PKC α and/or PKC β to a greater degree than does CaG; and/or
b) induce a greater amount of calcium flux than does CaG; and/or
c) induce a greater amount of chemotaxis of the cell than does CaG; and/or
d) activate a MAP kinase to a greater degree than does CaG,
is a peptide that is a more potent agonist of FPR than is CaG.

8. The method of claim 1, which is a method to identify a peptide that is an antagonist of FPR, wherein a candidate peptide that is shown to inhibit an FPR-mediated agonist activity is an antagonist of FPR.

9. The method of claim 1, wherein the peptide induces chemotactic activity of a human phagocyte.

10. The method of claim 1, wherein the peptide stimulates a proinflammatory response.

11. The method of claim 1, wherein the peptide inhibits an undesirable inflammatory response.

12. The method of claim 1, wherein the peptide stimulates an antimicrobial response.

13. The method of claim 1, wherein the peptide modulates an antibody response, wound healing, phagocytosis, the production of reactive oxygen intermediates, and/or the inhibition of bacterial infection.

14. The method of claim 1, which is high throughput.

15. A method for identifying an agent that enhances the agonist ability of CaG on FPR, comprising screening a candidate agent for its ability, when contacted with an FPR-expressing cell, in the presence of CaG, to
a) activate PKC ζ, PKC α and/or PKC β to a greater degree than does CaG; and/or
b) induce a greater amount of calcium flux than does CaG; and/or
c) induce a greater amount of chemotaxis of the cell than does CaG, and/or
d) activate a MAP kinase to a greater degree than does CaG.

16. A kit for carrying out a method of claim 1, comprising an FPR-expressing cell and means for detecting a CaG-FPR induced response.

17. An isolated complex comprising CaG and FPR.

18. A peptide identified by the method of claim 1.
